(19) United States
(12) Patent Application Publication
(10) Pub. No.: US 2004/0192953 A1
(43) Pub. Date: Sep. 30, 2004

(54) Process for the production of a diaryl carbonate

(76) Inventors: Claus-Peter Reisinger, Wixom, MI (US); Peter Fischer, Koln (DE); Sven-Michael Hansen, Leverkusen (DE); Marc Dahlmann, Dusseldorf (DE)

Correspondence Address: BAYER MATERIAL SCIENCE LLC, 100 BAYER ROAD, PITTSBURGH, PA, 15205, US

(21) Appl. No.: 10/791,587

(22) Filed: Mar. 2, 2004

(30) Foreign Application Priority Data
Mar. 7, 2003 (DE) 10309954.9

(51) Int. Cl. C07C 69/96

(52) U.S. Cl. 558/270

(57) ABSTRACT

A process for the production of an aromatic carbonate is disclosed. The process entails (i) tempering an amount of aromatic hydroxy compound for a period t2 at an average temperature T2 in a device by bringing it together with an amount B1 of component d) and an amount C1 of component e), to produce an activated aromatic hydroxy compound wherein component d) is at least one compound selected from the group consisting of the compounds of Ru, Os, Rh, Ir, Ni, Pd and of Pt, and wherein component e) is at least one compound selected from the group consisting of the compounds of Sc, Y, La, Ti, Zr, Hf, V, Nb, Ta, Cr, Mo, W, Mn, Fe, Ni, Co, Cu, Ag, Au, Zn, Cd, Hg, Ga, In, Tl, Ge, Sn, Sb, Bi and Pb, and (ii) reacting in a reaction apparatus for a time t1 and at an average temperature T1 the activated aromatic hydroxy compound with carbon monoxide and oxygen in the presence of a catalyst system comprising an amount B2 of component (d) and an amount C2 of component (e) to produce an aromatic carbonate, said t2 being less than 50% of t1 and said T2 being at least 15° C. below said T1.

### FIELD OF THE INVENTION

The invention relates to a process for the production of a diaryl carbonate (DAC) and more particularly to a catalytic process.

### SUMMARY OF THE INVENTION

A process for the production of an aromatic carbonate is disclosed. The process entails (i) tempering an amount of aromatic hydroxy compound for a period t2 at an average temperature T2 in a device by bringing it together with an amount B1 of component d) and an amount C1 of component e), to produce an activated aromatic hydroxy compound wherein component d) is at least one compound selected from the group consisting of the compounds of Ru, Os, Rh, Ir, Ni, Pd and of Pt, and wherein component e) is at least one compound selected from the group consisting of the compounds of Sc, Y, La, Ti, Zr, Hf, V, Nb, Ta, Cr, Mo, W, Mn, Fe, Ni, Co, Cu, Ag, Au, Zn, Cd, Hg, Ga, In, Tl, Ge, Sn, Sb, Bi and Pb, and (ii) reacting in a reaction apparatus for a time t1 and at an average temperature T1 the activated aromatic hydroxy compound with carbon monoxide and oxygen in the presence of a catalyst system comprising an amount B2 of component d) and an amount C2 of component e) to produce an aromatic carbonate, said t2 being less than 50% of t1 and said T2 being at least 15° C. below said T1.

### BACKGROUND OF THE INVENTION

The production of DAC by oxidative direct carbonylation of aromatic hydroxy compounds in the presence of carbon monoxide (CO), oxygen (O₂) and in the presence of a precious metal catalyst is known. Palladium is preferably used as the precious metal. A co-catalyst (e.g. manganese-, copper-, lead-, titanium-, or cobalt salts), a base, bromide sources, quaternary salts, various quinones or hydroquinones and dessicants can also be used. The process may be carried out in a solvent.

US-A 2002/0177724 discloses a multi-stage process for the production of diaryl carbonates from aromatic hydroxy compounds by reacting them with carbon monoxide and oxygen, a first stage of the process being carried out in the presence of carbon monoxide, but without the presence of oxygen.

EP-A 0 654 461 discloses, that carbonylation can be carried out reproducibly and with higher space-time yields, if the reaction mixture is “activated” with carbon monoxide i.e. purged with pure carbon monoxide for a certain period before the reaction is carried out.

However the process failed to deliver satisfactory yields for industrial application and produced large quantities of by-products. The object of the present invention is thus to provide a process that delivers a high yield and a high product quality, in other words purity of product.

A process was found, which allows a surprisingly high selectivity and yield. That means that the quantity of by-products is low and that the yield of the product is high. A fundamental characteristic of this process is that the catalyst system used is activated by thermal pre-treatment in a separate reaction apparatus.

### DETAILED DESCRIPTION OF THE INVENTION

The present invention thus provides, and the object of the invention is thus achieved by, a process comprising (i) tempering an amount of aromatic hydroxy compound for a period t2 at an average temperature T2 in a device by bringing it together with an amount B1 of component d) and an amount C1 of component e) to produce an activated aromatic hydroxy compound wherein component d) is at least one compound selected from the group consisting of the compounds of Ru, Os, Rh, Ir, Ni, Pd and of Pt, and wherein component e) is at least one compound selected from the group consisting of the compounds of Sc, Y, La, Ti, Zr, Hf, V, Nb, Ta, Cr, Mo, W, Mn, Fe, Ni, Co, Cu, Ag, Au, Zn, Cd, Hg, Ga, In, Tl, Ge, Sn, Sb, Bi and Pb, and

(ii) reacting in a reaction apparatus for a time t1 and at an average temperature T1 the activated aromatic hydroxy compound with carbon monoxide and oxygen in the presence of a catalyst system comprising an amount B2 of component d) and an amount C2 of component e) to produce an aromatic carbonate, said t2 being less than 50% of t1 and said T2 being at least 15° C. below said T1 and said aromatic hydroxy compound conforming to

R—O—H (II)

wherein R is an aromatic group.

The process according to the invention produces an aromatic carbonate of formula (I)

R—O—CO—O—R (I)

In the following, the terms “thermal pretreatment”, “thermal activation”, and “tempering” all refer to the same process step, i.e. the first step of the process according to the invention.

In a particular embodiment of the present invention, the stated device comprises several individual devices.

Other particular embodiments of the process according to the invention are described below.

The aromatic hydroxy compounds of formula (II)

R—O—H (II)

that may be reacted according to the invention include monohydroxy compounds such as phenol, o-, m- or p-cresol, o-, m- or p-chlorophenol, o-, m- or p-ethylphenol, o-, m- or p-propylphenol, o-, m- or p-methoxyphenol, 2,6-dimethyl phenol, 2,4-dimethyl phenol, 3,4-dimethyl phenol, 1-naphthol, 2-naphthol or di- or polyhydroxy compounds such as resorcinol and hydroquinone, as well as tris- and bisphenols such as 2,2-bis-(4-hydroxyphenyl)-propane (bisphenol A), 2,2-bis-(3,5-dimethyl-4-hydroxyphenyl)-propane, 1,1-bis-(4-hydoxyphenyl)-cyclohexane, 1,1-bis-(4-hydroxyphenyl)-3,3,5-trimethyl cyclohexane or 6,6′-dihydroxy-3,3,3′,3′-tetramethyl-1,1′-spiro(bis)-indane, 2,4′-hydroxybiphenyl or 4,4′-hydroxybiphenyl. Where there is substitution of the aromatic hydroxy compound, this is usually by 1 to 3 substituents with the meaning of C₁-C₁₈-alkyl, C₆-C₁₈-aryl, C₇-C₁₈-aralkyl, C₁-C₁₈-alkoxy, fluoro, chloro or bromo. Monohydroxy compounds are preferred, in particular phenol.

The catalyst component d) suitable for the process according to the invention contains preferably at lest one precious metal of Group VIII, preferably palladium. In the process according to the invention, it may be added in various forms. Palladium may be used in metallic form, e.g. as palladium black or on a support such as Pd/C, Pd/Al₂O₃, Pd/SiO₂ or preferably in the form of palladium compounds of oxidation stages 0 and +2, such as for example palladium(II)-acetyl acetonate, -halides, -carboxylates of C₂-C₁₈-carboxylic acids, -dicarboxylates such as oxalate, -nitrate, -sulfate, -oxide or palladium complexes, which may contain, for example, carbon monoxide, olefins, amines, nitriles, phosphorus compounds and halides. Palladium bromide and palladium acetyl acetonate are preferred in particular.

The quantity of catalyst component d) is not restricted in the process according to the invention. Preferably, enough catalyst is used to ensure that the concentration of the metal in the reaction charge is 1 to 3000 ppm, concentrations of 5 to 500 ppm being preferred in particular.

A metal of Groups IIIA, IIIB, IV A, IV B, V B, I B, II B, VI B, VII B, of the rare earth metals (atomic numbers 58-71) or the iron group of the periodic system of elements (Mendeleev), optionally also mixtures of these, may be used as catalyst component e) which acts as co-catalyst. The metal may be used in various oxidation stages. The documents U.S. Pat. No. 5,142,086, U.S. Pat. No. 5,231,210, U.S. Pat. No. 5,284,964, EP-A 0 350 697, EP-A 0 350 700 and U.S. Pat. No. 5,336,803 disclose the use of such elements as co-catalysts.

Pb, Ti, Mn, Cu, Co, V, Zn, Ce and Mo are preferred. Lead(U), manganese(II), manganese(III), copper(I), copper (II), cobalt(II), cobalt (III), vanadium(III) and vanadium (IV) are mentioned, without restricting the process according to the invention. The metals may be used for example as halides, oxides, carboxylates of C₂-C₁₈-carboxylic acids, diketonates or nitrates and also as complex compounds, which may contain, for example, carbon monoxide, olefins, aromatic and aliphatic monoamines or polyamines, phosphorus compounds, pyridine, bipyridine, terpyridine, quinoline, isoquinoline, cryptands, Schiff bases and halides. Mn, Cu, Mo, Ti, Pb and Ce are preferred in particular. Manganese compounds are preferred most particularly in the process according to the invention, manganese(II)-, and manganese(III)-complexes being preferred in particular and manganese(II)acetyl acetonate or manganese(III)-acetyl acetonate being preferred most particularly, as well as manganese(II)bromide.

The catalyst component e) (co-catalyst), which may also be formed in situ, is preferably added in such a quantity that its concentration is in the range of 0.0001 to 20 wt. % of the reaction mixture. The concentration range of 0.001 to 5 wt. % is preferred, 0.005 to 2 wt. % is preferred in particular.

Bromide compounds (V-a), bases (V-b) or solvents (V-c) for example, may be used as optional components f) of the catalyst system.

The bromide compounds (V-a) used in the context of the present invention are, for example, the alkali bromides or earth alkali bromides, but preferably the bromide salts of organic cations. The organic cations may be, for example, ammonium-, guanidinium-, phosphonium-, or sulfonium salts substituted with organic groups, optionally also mixtures of these. Ammonium-, guanidmium-, phosphonium-, and sulfonium salts that contain, as organic groups, C₆- to C₁₀-aryl, C₇- to C₁₂-aralkyl- and/or C₁- to C₂₀-alkyl groups are suitable for use in the process according to the invention. Ammonium salts that carry, as organic groups, C₆- to C₁₀-aryl-, C₇- to C₁₂-aralkyl- and/or C_{1- to C}₂₀-alkyl groups, are preferred in the process according to the invention, tetrabutyl ammonium bromide and tetrabutyl phosphonium bromide being preferred in particular. The quantity of such a quaternary salt may be, for example, 0.1 to 20 wt. % in relation to the weight of the reaction mixture. This quantity is preferably 0.5 to 15 wt. %, in particular 1 to 5 wt. %.

The bases (V-b) that may be used for the process according to the invention are, for example, alkali hydroxides, alkali salts or quaternary salts of weak acids such as alkali tert-butylates or alkali salts or quaternary salts of aromatic hydroxy compounds of formula (II), in which R has the meaning given above. An alkali salt or quaternary salt of the aromatic hydroxy compound of formula (II), which is converted during the process according to the invention to the aromatic carbonate of formula (I), for example tetrabutyl aummonium- or potassium phenolate, is preferred most particularly.

The alkali salts can be lithium-, sodium-, potassium, rubidium-, or caesium salts. Lithium-, sodium- and potassium phenolate are preferred, potassium phenolate is preferred in particular.

The quaternay salts may be ammonium-, phosphonium-, pyridinium-, sulfonium- or guanidinium salts, which have, as organic groups, C₆- to C₁₈-aryl, C₇- to C₁₈-aralkyl- and/or C₁- to C₂₀-alkyl groups. The groups may all be the same or different, mixtures of several quaternary salts may optionally also be used. Preferably the same cation is optionally used here as is used as the bromide for component (V-a). Furthermore, Tetraphenyl phosphonium, tetrabutyl ammonium-, tetrabutyl phosphonium are preferred and tetrabutyl ammonium is preferred in particular.

Alternatively, trialkyl amine bases, such as tributyl amine, diisopropyl ethyl amine, 1,5-diazabicyclo[4.3.0]non-5-ene (DBN) or 1,8-diazabicyclo[5.4.0]undec-7-ene (DBU) may be used.

The base (V-b) is added in a quantity dependent on the stoichiometry. The ratio of platinum metal, e.g. palladium, to base is preferably selected so that 0.1 to 5000, preferably 1 to 1000, in particular 10 to 300 equivalent bases are used per mol of platinum metal, e.g. palladium.

Inert solvents (V-c) may optionally be used under the reaction conditions. Examples of solvents are aliphatic hydrocarbons such as pentane, petroleum ether, cyclohexane, isooctane, aromatic hydrocarbons such as benzene, toluene, xylenes, chloro aromatics such as chlorobenzene or dichlorobenzene, ethers, such as dioxane, tetrahydrofuran, t-butyl-methyl ether, anisol, amides such as dimethyl acetaride, N-methyl pyrrolidinone, alcohols such as t-butanol, cumyl alcohol, isoamyl alcohol, diethylene glycol, tetramethyl urea. Mixtures of solvents may be used. The reaction mixture may contain a proportion of 1 to 99%, preferably 20 to 98%, in particular 30 to 98% of the inert solvent. Particularly when using inorganic components V-a or V-b, it is beneficial to use solvents that promote the solubility of inorganic salts such as NaBr or NaOPh, such as e.g. dipolar, aprotic solvents (e.g. N,N-dimethyl formamide, N,N-dimethyl acetamide, N-methyl pyrrolidinone, sulfolane, acetonitrile) or crown ethers, cryptands or “open crown ethers” (e.g. diethylene glycol dimethyl ether, triethylene glycol dimethyl ether, tetraethylene glycol dimethyl ether).

In the process according to the invention, the catalyst system is thermally activated by thermal pre-treatment of mixtures that contain, independently of each other, proportions of about 1 to 100% of the total quantity of each of the components a), d), e) and optionally f). This means that about I to 100% of each component is activated according to the first step of the inventive process. This also means that the thermally activated proportion of the total quantity of one component is independent from the thermally activated proportion of the total quantity of another component.

The largest possible proportion of component d) should preferably be selected to be greater than about 50 wt. % of the total quantity, particularly preferably greater than about 80 wt. %, most particularly preferably about 90 to 100 wt. % of the total quantity of component d). The largest possible proportion of component e) should preferably be selected to be greater than about 50 wt. % of the total quantity, particularly preferably greater than about 80 wt. %, most particularly preferably about 90 to 100 wt. % of the total quantity of component e). The proportion of component (V-a) is preferably about 1 to 100 wt. %, particularly preferably about 5 to 100 wt. %.

The relative proportions of the individual components may be changed during activation by charging at least one component. The relative amounts of the components thermally activated according to the invention may therefore be on average about 1 to 100% of the total weight of the reaction mixture. To keep the dimensions of the reaction apparatus as small as possible, it is preferable if only weights of less than 75% of the total weight, particularly preferably less than 40% of the total weight are charged into the one or more devices, activation apparatus(es) (i.e. the device) and the remaining quantity is introduced only into the subsequent reaction apparatus(es).

During activation CO and O₂ and optionally other carrier gases may be used within the bounds of the restrictions of the explosion limits, in any ratio to each other. The CO/O₂ ratio during activation is preferably approximately the same or greater than the ratio used in the reaction. The pressure is preferably about 0.01 to 500 bar. A pressure lower than or equal to the average reaction pressure is preferably used.

The period t2 in which the thermal activation according to the invention takes place is normally about 1 minute to 10 hours and comprises a proportion of less than 50%, preferably less than 35%, of the following total reaction time t1 or average residence time.

Activation is carried out at temperatures T2 that are on average more than 15° C., preferably more than 25° C., in particular more than 35° C., below the average reaction temperature T1.

According to the invention, the average activation temperature T2 is defined as follows. In a plot showing the activation temperature against the activation period (or residence time), the integral of the activation temperature-activation period curve is formed. The average activation temperature is then given by the quotient of this integral and the activation period.

According to the invention, the average reaction temperature T1 is defined as follows. In a plot showing the reaction temperature against the reaction period (or residence time), the integral of the reaction temperature-reaction period curve is formed. The average reaction temperature is then given by the quotient of this integral and the reaction period.

The average activation temperature T2 is normally in the range of about 10 to 120° C.

Various temperature profiles are possible. Activation may be carried out at a constant temperature, at a range of different temperatures, for example a step function rising in equidistant steps or steps of differing length or various other linear, convex or concave temperature-time profiles. Various such time-temperature profiles may be combined into an overall profile. Constant temperatures, or temperature-time profiles with a monotonic positive gradient, are preferred. The final temperature during tempering preferably differs from the starting temperature of the reaction by less than 5° C.

As the activation stage is normally followed immediately by the reaction, the final temperature of activation is typically also the starting temperature of the reaction, which is normally kept constant by known means. According to the invention, constant temperature is therefore understood to be a combination of one temperature-time profile with the gradient 0 and a heating-up phase, i.e. an increase from this selected constant temperature to the starting temperature of the reaction that is monotonic within the limits of the controls.

The process according to the invention may be carried out both continuously and discontinuously. The two steps of the process, i.e. the activation and the reaction, are carried out in two separate devices. When carried out discontinuously, activation takes place in a separate activation apparatus (herein also called device), e.g. a vessel with a gas intake stirrer. From this device the activated mixture is dosed before or—batchwise or continuously—during the reaction. When carrying out the process continuously, the mixture intended for activation is preferably fed through a suitable device (for example a stirred vessel, a bubble column or a combination of one or more nozzles and a tube), the dimensions of which are such that the average residence time is less than 50% of the residence time of the reaction apparatus.

The process according to the invention for carbonate formation is preferably carried out at an average reaction temperature T1 of 30 to 200° C., preferably 50 to 150° C., in particular 60 to 130° C., and preferably at an average reaction pressure of 1 to 200 bar, preferably 1 to 50 bar, in particular 1 to 10 bar. The process is preferably carried out at a constant pressure or a time-pressure step profile with various constant pressures or various constant pressure gradients. The process is preferably carried out at a constant temperature or a time-temperature step profile with various constant temperatures or various constant temperature gradients.

The composition of the reaction gases carbon monoxide and oxygen may be varied in the reaction phase within broad concentration limits, however a CO:O₂ molar ratio (standardised to CO) of 1:0.001 to 1:1, preferably 1:0.01 to 1:0.5 and in particular 1:0.02 to 1:0.3 is preferably set. The oxygen partial pressure at these molar ratios is high enough to allow high space-time yields to be achieved.

All starting compounds may be contaminated with impurities from their production and storage, however for the purity of the end product it is desirable to work with chemicals that are as clean as possible. The reaction gases are not subject to any particular purity requirements either. Thus synthesis gas may be used as a source of CO and air as an O₂ carrier, however care must be taken to ensure that no catalyst poisons such as e.g. sulfur or its compounds are introduced. The gases may be rarefied with one or more other gases such as nitrogen, argon, carbon dioxide or hydrogen. In the preferred embodiment of the process according to the invention, pure CO and pure oxygen are used.

The catalyst system may be either heterogeneous or homogenous. In a heterogeneous catalyst system either one of the catalyst components d) or e) or both components d) and e) are applied to a heterogeneous support. Heterogeneous catalyst systems may be used in the form of a powder or molded bodies. If only catalyst component d) is heterogeneous, the co-catalyst (component e) is dissolved homogeneously in the reaction solution. The quantity of the platinum metal in the total weight of the heterogeneous catalyst is 0.01 to 15 wt. %, preferably 0.05 to 10 wt. %, calculated as platinum metal. The remaining optional components of the catalyst system (i.e. components f), such as the bases, the quaternary compound, are still dissolved homogeneously in the reaction solution.

The quantity of the co-catalyst in the total weight of the heterogeneous catalyst is preferably 0.01 to 15 wt. %, more preferably 0.05 to 10 wt. %, calculated as metal.

Suitable catalyst supports are one or more metal oxides from the group of V, Mn, Ti, Cu, Zr, La of the rare earth metals (atomic numbers 58-71), both in the sense of chemically uniform pure substances and in mixture, and also iron- and cobalt oxides, nickel-, aluminium-, silicon- and magnesium oxide, zeolites and activated carbons. If the supported catalyst is used as a powder, the stirred vessels to be used to mix the reaction components are designed to be equipped with stirrers that may be used for this purpose, or as a bubble column reactor.

When working with supported catalyst powders as a suspension in stirred vessels or bubble columns, quantities of 0.001 to 50 wt. %, preferably of 0.01 to 20 wt. %, in particular of 0.1 to 10 wt. % supported catalyst powder are preferably used in relation to the quantity of aromatic hydroxy compound used.

In preferred embodiments, the heterogeneous supported catalyst is placed so that it is stationary in stirred vessels, a bubble column, a trickle phase reactor or cascades of these reactors. Separation of the supported catalyst is then completely unnecessary. Stirred tanks, autoclaves and bubble columns are suitable as reactors for the process according to the invention with homogeneous or heterogeneous catalysts and may be used as single reactors or as a cascade. 2 to 15, preferably 2 to 10, in particular 2 to 5 reactors may be placed one after the other as a cascade.

The stirred vessels preferably used according to the invention to mix the reaction components are equipped with stirrers suitable for this purpose. Such stirrers are known to the person skilled in the art. Examples are: disc, impeller, propeller, paddle, MIG and intermig stirrers, tube stirrers and various types of hollow stirrer. Preferred stirrers are those that allow effective mixing of gases and liquids, for example hollow tube gas dispersion stirrers, propeller stirrers etc.

The following types of bubble column may be used in the process according to the invention: simple bubble columns, bubble columns with inserted components, such as e.g. bubble columns with parallel chambers, cascade bubble columns with sieve plates or single-hole plates, bubble columns with packing, with static mixers, pulsating sieve plate bubble columns, loop reactors such as e.g. mammoth loop reactors, downflow loop reactors, jet loop reactors, free jet reactors, jet nozzle reactors, bubble columns with liquid immersion jets, upflow-downflow bubble columns and other bubble column reactors known to the person skilled in the art. Such apparatus is disclosed for example in Chem. Ing. Tech. 51 (1979) No. 3, p. 208-216 and in W.-D. Deckwer, Reaktionstechnik in Blasensaiulen, Otto Salle Verlag 1985.

In a preferred embodiment bubble column reactors and bubble column cascades are used, which allow effective mixing of gas and liquids, such as for example cascade bubble columns and loop reactors. To maintain good intermixing of liquid and reaction gas, distribution and re-dispersion organs may be fitted along the longitudinal axis of the bubble column reactors. Possible fixed re-dispersion organs are single-hole plates, perforated plates, sieve plates, and other inserted components known to the person skilled in the art. The conventional devices such as porous sintered plates, perforated plates, sieve plates, insertion tubes, nozzles, gas dispersion rings and other dispersion devices known to the person skilled in the art may be used for initial dispersion of the reaction gas in the liquid phase when dosing in.

The process according to the invention may include variants. One possibility is the particular form of the discontinuous process described below. In this process, CO and oxygen are fed into the mixture to be activated comprising components a), d), e) and optionally f) either through a gas dispersion stirrer as in the case of a stirred tank or through other known gas distribution organs. Once the optimum conversion is reached in the reaction stage, i.e. the second step of the process according to the invention, the reaction mixture is removed from the reactor or optionally processed, e.g. filtered in the reactor. When using supported catalysts in powder form, they may be separated from the reaction mixture e.g. by filtration, sedimentation or centrifuging.

Where the charge is the same, supported catalysts used in discontinuous runs may be re-used repeatedly, optionally without cleaning. When working continuously, the supported catalysts used may remain in the reactor over a long period of time and may optionally be regenerated.

A continuous process in a single reactor or a cascade of several reactors is preferably used. When using stationary heterogeneous catalysts, they may remain in the reactor for a long period of time and may optionally also be regenerated there.

### EXAMPLES

The reaction components were analysed by gas chromatography (GC), the masses of the components being measured by means of an internal standard. Mean values and standard deviations were determined from the data obtained at intervals of several hours. The selectivity was calculated by adding the quantity of residual phenol contained in the reaction mixture and the phenol converted to diphenyl carbonate (DPC) and dividing this by the total quantity of phenol used. The phenol not recorded by this method was converted into by-products.

### Example 1 (Ex. 1)

A reaction stream composed of 0.05 g/h palladium bromide, 0.2 g/h manganese trisacetyl acetonate, 10 g/h tetrabutyl ammonium bromide, 9 g/h tetrabutyl ammonium phenolate, 79.95 g/h phenol, 8.8 g/h GC-standard and 241 g/h chlorobenzene was dosed from three different receiving flasks (1^{st} receiving flask: palladium bromide, tetrabutyl ammonium bromide, chlorobenzene, 2^{nd} receiving flask: tetrabutyl ammonium bromide, tetrabutyl ammonium phenolate, phenol, chlorobenzene, ₃^{rd} receiving flask: manganese trisacetyl acetonate, chlorobenzene) into a catalyst activating vessel (RA1) at a temperature of 50° C. and an average residence time of 43 minutes. It was purged with 100 Ni/h (100 standard litres per hour) of a gas mixture of carbon monoxide and oxygen (99:1 vol. %) at a pressure of 3 bar. A partial quantity was then dosed into the outlet from the third receiving flask, so that a total stream composed of 0.05 g/h palladium bromide, 0.8 g/h manganese trisacetyl acetonate, 10 g/h tetrabutyl ammonium bromide, 9 g/h tetrabutyl ammonium phenolate, 79.95 g/h phenol, and 391.3 g/h chlorobenzene resulted. This stream was fed into a stirred reaction vessel (RA2), which was purged with 300 Nl/h of a gas mixture of carbon monoxide and oxygen (97:3 vol. %) at a pressure of 3 bar. The process was carried out at a reaction temperature of 100° C. and an average residence time of 127 minutes. After the process had reached the steady state, GC samples were taken from the outlet of the reaction apparatus (RA2) and the outlet of the catalyst activation vessel (RA1). The results are given in Table 1.

### Reference Example (Ref. 1)

A reaction stream composed of 0.05 g/h palladium bromide, 0.8 g/h manganese trisacetyl acetonate, 10 g/h tetrabutyl ammonium bromide, 9 g/h tetrabutyl ammonium phenolate, 80.03 g/h phenol, and 391.2 g/h chlorobenzene was fed from three different receiving flasks (1^{st} receiving flask: palladium bromide, tetrabutyl ammonium bromide, chlorobenzene, 2^{nd} receiving flask: tetrabutyl ammonium bromide, tetrabutyl ammonium phenolate, phenol, chlorobenzene, 3^{rd} receiving flask: manganese trisacetyl acetonate, chlorobenzene) into a stirred reaction vessel (RA2), which was purged with 300 Nl/h of a gas mixture of carbon monoxide and oxygen (97:3 vol. %) at a pressure of 3 bar. The process was carried out at a reaction temperature of 100° C. and an average residence time of 129 minutes. After the process had reached the steady state, GC samples were taken from the outlet of the reaction apparatus (RA2). The results averaged over the following reaction period are given in Table 1.

TABLE 1
Comparison of selectivities and DPC yields

| | Reactor | Phenol | |
|---|---|---|---|
| | sampling site | selectivity [%] | DPC [wt. %] |
| Ex. 1 | RA 1 | | 0.33 ± 0.02 |
| | RA 2 | 94.0 ± 0.2 | 7.59 ± 0.30 |
| Ref. 1 | RA 2 | 87.9 ± 2.4 | 5.95 ± 0.25 |

It is found that, although hardly any reaction takes place in the separate catalyst activation vessel (RA1), both the selectivity and the catalyst activity are higher, at the same residence time, in the outlet of the reactor (RA2).

Although the invention has been described in detail in the foregoing for the purpose of illustration, it is to be understood that such detail is solely for that purpose and that variations may be made therein by those skilled in the art without departing from the spirit and scope of the invention except as it may be limited by the claims.

What is claimed is:

1. A process for the production of an aromatic carbonate comprising
(i) tempering an aromatic hydroxy compound of formula (II)
R—O—H (II)
wherein R represents an aromatic group, for a period t2 at an average temperature T2 in a device by bringing it together with an amount B1 of component d) and an amount C1 of component e) , to produce an activated aromatic hydroxy compound wherein component d) is at least one compound selected from the group consisting of the compounds of Ru, Os, Rh, Ir, Ni, Pd and of Pt, and wherein component e) is at least one compound selected from the group consisting of the compounds of Sc, Y, La, Ti, Zr, Hf, V, Nb, Ta, Cr, Mo, W, Mn, Fe, Ni, Co, Cu, Ag, Au, Zn, Cd, Hg, Ga, In, Tl, Ge, Sn, Sb, Bi and Pb, and
(ii) reacting in a reaction apparatus for a time t1 and at an average temperature T1 the activated aromatic hydroxy compound with carbon monoxide and oxygen in the presence of a catalyst system comprising an amount B2 of component (d) and an amount C2 of component (e) to produce an aromatic carbonate said t2 being less than 50% of t1 and said T2 being at least 15° C. below said T1.

2. The process according to claim 1 wherein the amount B1 is 50 to 100% of the total weight of B1 and B2 and the amount C1 is 10 to 100% of the total weight of C1 and C2.

3. The process according to claim 1 wherein the reacting in a reaction apparatus takes place in the presence of a solvent.

4. The process according to claim 1 wherein the catalyst system additionally contains at least one component selected from the group consisting of bromides, bases and solvents.

5. The process according to claim 1 wherein t2 is less than 35% of t1.

6. The process according to claim 1 wherein T2 is at least 25° C. below T1.

7. The process according to claim 1 wherein tempering is carried out at a constant temperature T2.

8. The process according to claim 1 wherein tempering is carried out at a temperature-time profile with a monotonic positive gradient.

9. The process according to claim 1 wherein the temperature of the tempered aromatic compound at the end of (i) differs from the temperature of the tempered aromatic compound at the start of (ii) by less than 5° C.

10. The process according to claim 1 wherein the aromatic carbonate is diphenyl carbonate.

11. The process according to claim 1 wherein the process is carried out continuously.

12. The process according to claim 11, wherein the mass stream that flows through the second device is less than 75% of the mass stream that flows through the reaction apparatus.
